(19) United States
(12) Patent Application Publication
(10) Pub. No.: US 2022/0395540 A1
(43) Pub. Date: Dec. 15, 2022

(54) TREATMENT OF COVID-19 LUNG INJURY USING UMBILICAL CORD PLASMA BASED COMPOSITIONS

(71) Applicant: THERAPEUTIC SOLUTIONS INTERNATIONAL, INC., OCEANSIDE, CA (US)

(72) Inventors: James Veltmeyer, Oceanside, CA (US); Thomas E. Ichim, Oceanside, CA (US); Timothy G. Dixon, Oceanside, CA (US)

(73) Assignee: THERAPEUTIC SOLUTIONS INTERNATIONAL, INC., OCEANSIDE, CA (US)

(21) Appl. No.: 17/343,712

(22) Filed: Jun. 9, 2021

(51) Int. Cl. A61K 35/51; A61P 37/06; A61P 11/00; A61K 31/47; A61K 31/436; A61K 47/18; C12N 15/113; A61K 31/7088; A61K 45/06

(52) U.S. Cl. CPC A61K 35/51; A61P 37/06; A61P 11/00; A61K 31/47; A61K 31/436; A61K 47/18; C12N 15/113; A61K 31/7088; A61K 45/06; C12N 2310/11; C12N 2320/31; C12N 2310/14; C12N 2310/122; C12N 2310/531

(57) ABSTRACT

Disclosed are means, methods and compositions of matter useful for treatment of lung inflammation associated with viral and bacterial infections, as well as with systemic inflammation, through administration of umbilical cord blood derived plasma-based compositions. In one embodiment the invention teaches administration of umbilical cord blood plasma together with pterostilbene, and/or sulforaphane, and/or thymoquinone, and/or Epigallocatechin gallate (EGCG) and/or n-acetylcysteine in an aerosolized manner to patients suffering from COVID-19 associated pulmonary deficiencies. In another embodiment, umbilical cord blood plasma is administered with immune stimulatory agents in order to concurrently inhibit propagation of viral load in the lung while suppressing pulmonary deficiencies.

### CROSS-REFERENCE TO RELATED APPLICATIONS

This application claims the benefit of priority to U.S. Provisional Application No. 63/023,217, filed May 11, 2020, the contents of which are hereby incorporated by reference.

### FIELD OF THE INVENTION

The present invention pertains to the treatment of pulmonary inflammation, such as from COVID-19, through the use of umbilical cord plasma based compositions.

### BACKGROUND

Acute respiratory distress syndrome (ARDS) is a life-threatening medical condition mainly known by widespread and uncontrolled inflammation it causes in the lungs, which in turn is associated with the loss of surfactant and impaired pulmonary capillary endothelium, resulting in fluid accumulation in the distal airspaces. Several etiologies can trigger ARDS, including sepsis, primary respiratory infections, and significant trauma. To this day, ARDS continues to result in high mortality and high costs related to usually prolonged hospitalization in the intensive care unit (ICU), stressing hospital resources [1]. Furthermore, ARDS survivors often have long-term pulmonary, neuromuscular, and cognitive symptoms and a diminished quality of life [2].

An acute respiratory syndrome, lung failure, and fulminant pneumonia are major lung diseases present in viral infections of current interest, some viral infections also cause extrapulmonary diseases including rhabdomyolysis and encephalopathy through cytokine storms [12,13]. Also, dealing with the antiviral resistance and secondary infection-induced multiple organ dysfunction in patients is still a serious concern, and there is an exigent demand to explore an effective strategy against this affection [14].

Treatment is currently limited to supportive care and appropriate use of mechanical ventilation and fluid management, since a number of different pharmacologic therapies have failed to demonstrate benefit[3, 4]. Extracorporeal membrane oxygenation (ECMO) has been used in patients with severe ARDS; however, in a recent systematic review and meta-analysis of current evidence, no association with improved outcomes could be demonstrated in adult patients [5]. As such, new therapeutic approaches are needed.

Adoptive transfer of mesenchymal stromal cells (MSCs) ameliorates experimentally induced acute lung injury in preclinical animal models and in ex vivo perfused human lungs [6-14]. Although not completely understood, the mechanisms of MSC actions in acute models of ARDS include the release of paracrineanti-inflammatory and antibacterial peptides and mitochondrial transfer through cell—cell contact with damaged alveolar epithelial cells in the absence of permanent cell engraftment [9, 15-17]. Furthermore, MSCs release extracellular vesicles (EVs), which have reduced inflammation and promote tissue regeneration in different preclinical models [6, 18-20].

The novel coronavirus disease 2019 (COVID-19) has become a global public health emergency since patients were first detected in Wuhan, China, in December 2019. The number of COVID-19 confirmed patients have sharply increased worldwide in countries including Germany, South Korea, Vietnam, Singapore, Italy, and USA (21). At this point in time, no specific drugs or vaccines are available to effectively treat the patients with COVID-19 infection. Hence, there is a large unmet need for a safe and effective treatment for COVID-19 infected patients, especially in the most severe cases.

Several reports have shown that the initial step of the HCoV-19 pathogenesis is that the virus specifically recognizes the angiotensin I converting enzyme 2 receptor (ACE2) by its spike protein(22-24). ACE2-positive cells are infected by the HCoV-19, much like SARS-2003 (25, 26). In addition, a research team from Germany revealed that the cellular serine protease TMPRSS2 for HCoV-19 Spike protein priming is also essential for the host cell entry and spread (27), like the other coronavirus (i.e. SARS-2003) (28, 29).

Unfortunately, the ACE2 receptor is widely distributed on the human cells surface, especially the alveolar type II cells (AT2) and capillary endothelium (30), and the AT2 cells highly express TMPRSS2 (29). However, immune cells, such as T and B lymphocytes, and macrophages in the bone marrow, lymph nodes, thymus, and the spleen, are consistently negative for ACE2(30).

These findings suggest that immunological therapy may be used to treat the infected patients. However, the immunomodulatory capacity may be not be strong enough, if only one or two immune factors were used, as the virus can stimulate a massive cytokine storm in the lung, such as IL-2, IL-6, IL-7, GSCF, IP10, MCP1, MIP1A, and TNFα, followed by edema, air exchange dysfunction, acute respiratory distress syndrome, acute cardiac injury and secondary infection(31), which may lead to death. Therefore, avoiding the cytokine storm may be the key for the treatment of HCoV-19 infected patients.

MSCs have been widely used in cell-based therapy, from basic research to clinical trials (32, 33). Safety and effectiveness have been clearly documented in many clinical trials, especially in the immune-mediated inflammatory diseases, such as graft versus-host disease (GVHD) (34) and systemic lupus erythematosus (SLE)(35). MSCs play a positive role mainly in two ways, namely immunomodulatory effects and differentiation abilities(36). MSCs can secrete many types of cytokines by paracrine secretion or make direct interactions with immune cells, leading to immunomodulation(37). The immunomodulatory effects of MSCs are triggered further by the activation of TLR receptor in MSCs, which is stimulated by pathogen-associated molecules such as LPS or double-stranded RNA from virus (38, 39), like the HCoV-19.

Unfortunately, MSCs possess a variety of drawbacks such as cost of manufacturing, as well as potential for evoking allogeneic immune reactions. There is a need for new treatment approaches to ARDS.

### SUMMARY

Preferred embodiments are directed to a method of treating pulmonary inflammation comprising the steps of: a) identifying a patient with pulmonary inflammation; b) administering cord blood plasma and/or serum together with one or more adjuvants; and c) assessing pulmonary inflammation in order to determine whether adjustment of dosage is necessary.

Preferred methods include embodiments wherein said pulmonary inflammation is acute respiratory distress syndrome.

Preferred methods include embodiments wherein said pulmonary inflammation is pneumonia.

Preferred methods include embodiments wherein said pulmonary inflammation is enhanced number of neutrophils in the alveolar area.

Preferred methods include embodiments wherein said pulmonary inflammation is enhanced activation of neutrophils in the alveolar area.

Preferred methods include embodiments wherein said pulmonary inflammation is enhanced number of mast cells in the alveolar area.

Preferred methods include embodiments wherein said pulmonary inflammation is enhanced activation of mast cells in the alveolar area.

Preferred methods include embodiments wherein said pulmonary inflammation is enhanced number of macrophages in the alveolar area.

Preferred methods include embodiments wherein said pulmonary inflammation is enhanced activation of macrophages in the alveolar area.

Preferred methods include embodiments wherein said pulmonary inflammation is enhanced number of T cells in the alveolar area.

Preferred methods include embodiments wherein said pulmonary inflammation is enhanced activation of T cells in the alveolar area.

Preferred methods include embodiments wherein said activation of neutrophils is production of neutrophil elastase.

Preferred methods include embodiments wherein said activation of neutrophils is production of reactive oxygen radicals.

Preferred methods include embodiments wherein said activation of neutrophils is release of DNA extracellular traps.

Preferred methods include embodiments wherein said activation of neutrophils is release of matrix metalloproteases.

Preferred methods include embodiments wherein said activation of macrophages is release of matrix metalloproteases.

Preferred methods include embodiments wherein said activation of macrophages is release of nitric oxide.

Preferred methods include embodiments wherein said activation of macrophages is release of reactive oxygen radicals and/or TNF-alpha.

Preferred methods include embodiments wherein said activation of T cells is release of interleukin 2.

Preferred methods include embodiments wherein said activation of T cells is release of interleukin 17.

Preferred methods include embodiments wherein said activation of T cells is release of granzyme.

Preferred methods include embodiments wherein said activation of T cells is release of perforin.

Preferred methods include embodiments wherein said pulmonary inflammation is associated with activation of the complement cascade.

Preferred methods include embodiments wherein said cord blood plasma/serum is derived from umbilical cord at term delivery.

Preferred methods include embodiments wherein said cord blood plasma/serum is derived from umbilical cord at pre-term delivery.

Preferred methods include embodiments wherein said cord blood plasma/serum is fractionated to obtain fractions enriched in exosomes.

Preferred methods include embodiments wherein said exosomes are of a size between 60-200 nanometers.

Preferred methods include embodiments wherein said adjuvant is an anti-oxidant.

Preferred methods include embodiments wherein said anti-oxidant is selected from a group comprising of: antioxidant is selected from a group comprising of: ascorbic acid and derivatives thereof, alpha tocopherol and derivatives thereof, rutin, pterostilbene, quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, and superoxide dismutase.

Preferred methods include embodiments wherein said pulmonary inflammation is caused by a cytokine storm caused by excessive production of cytokines.

Preferred methods include embodiments wherein said excessive production of cytokines is mediated by unrestrained activation of monocytes.

Preferred methods include embodiments wherein said excessive production of cytokines is mediated by unrestrained activation of peripheral blood mononuclear cells.

Preferred methods include embodiments wherein said excessive production of cytokines is mediated by unrestrained activation of dendritic cells.

Preferred methods include embodiments wherein said excessive production of cytokines is mediated by unrestrained activation of gamma delta T cells.

Preferred methods include embodiments wherein said excessive production of cytokines is mediated by unrestrained activation of natural killer cells.

Preferred methods include embodiments wherein said production of cytokines is not significantly controlled by anti-inflammatory cytokines.

Preferred methods include embodiments wherein a virus generates factors which inhibit the body from suppressing production of inflammatory cytokines.

Preferred methods include embodiments wherein said excessive production of cytokines is production of cytokines selected from a group comprising of: a) MCP-1; b) interleukin 1 beta; c) interleukin 6, d) interleukin 8; e) interleukin 11; f) interleukin-18; g) interleukin-21; h) interleukin 27; i) interleukin 33; j) HMGB-1; and k) TNF-alpha.

The method of claim 31, wherein said excessive production of cytokines is Th1/Th17 cytokine storm.

Preferred methods include embodiments wherein said cytokine storm occurs as a result of a viral infection.

Preferred methods include embodiments wherein said viral infection is a coronavirus infection.

Preferred methods include embodiments wherein said coronavirus infection is an infection with SARS-Cov-2.

Preferred methods include embodiments wherein reduction of excessive cytokine production is further achieved by co-administration of hydroxychloroquine and/or chloroquine.

Preferred methods include embodiments wherein reduction of excessive cytokine production is further achieved by co-administration of rapamycin.

Preferred methods include embodiments wherein reduction of excessive cytokine production is further achieved by co-administration of rapamycin.

Preferred methods include embodiments wherein reduction of excessive cytokine production is further achieved by co-administration of a chelating agent.

Preferred methods include embodiments wherein said chelating agent is deferoxamine mesylate.

Preferred methods include embodiments wherein reduction of excessive cytokine production is further achieved by co-administration of an agent capable of inhibiting NF-kappa B.

Preferred methods include embodiments wherein said NF-kappa B is inhibited by antisense oligonucleotides targeting one or more sequences found in NF-kappa B components.

Preferred methods include embodiments wherein said NF-kappa B is inhibited by RNA interference targeting one or more sequences found in NF-kappa B components.

Preferred methods include embodiments wherein said RNA interference is induced by administration of short interfering RNA.

Preferred methods include embodiments wherein RNA interference is induced by administration of short hairpin RNA.

Preferred methods include embodiments wherein said agent capable of inhibiting NF-kappa B activation is a decoy oligonucleotide.

Preferred methods include embodiments wherein said agent capable of inhibiting NF-kappa B activation is selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).

Preferred methods include embodiments wherein an immune suppressive agent is administered in combination with said cord blood/sera and adjuvant combinations.

Preferred methods include embodiments wherein said immune suppressive agent is selected from a group comprising of: cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), Monoclonal antibodies, basiliximab (Simulect), daclizumab (Zinbryta)

Preferred methods include embodiments wherein said adjuvant added to said cord blood plasma/serum is one or more antiviral agents.

Preferred methods include embodiments wherein said antiviral agent is chloroquine.

Preferred methods include embodiments wherein said antiviral agent is hydroxychloroquine.

Preferred methods include embodiments wherein said antiviral agent is remdesivir.

Preferred methods include embodiments wherein said antiviral agent is lopinavir.

Preferred methods include embodiments wherein said antiviral agent is Reproxalap.

Preferred methods include embodiments wherein said antiviral agent is Apabetalone.

Preferred methods include embodiments wherein said antiviral agent is Tradipitant.

Preferred methods include embodiments wherein said antiviral agent is Arbidol umifenovir.

Preferred methods include embodiments wherein said antiviral agent is Ganovo danoprevir.

Preferred methods include embodiments wherein said antiviral agent is Riavax tertomotide.

Preferred methods include embodiments wherein said antiviral agent is Thymosin alpha 1.

Preferred methods include embodiments wherein said antiviral agent is Ifenprodil (NP-120).

Preferred methods include embodiments wherein said antiviral agent is Avigan favipiravir.

Preferred methods include embodiments wherein said antiviral agent is Aviptadil

Preferred methods include embodiments wherein said antiviral agent is Oseltamivir.

Preferred methods include embodiments wherein said pulmonary inflammation is associated with viral infection.

Preferred methods include embodiments wherein said pulmonary inflammation is associated with bacterial infection.

Preferred methods include embodiments wherein said pulmonary inflammation is associated with fungal infection.

Preferred methods include embodiments wherein said pulmonary inflammation is associated with sepsis.

Preferred methods include embodiments wherein said pulmonary inflammation is associated with multiple organ failure.

Preferred methods include embodiments wherein said pulmonary inflammation is associated with trauma.

Preferred methods include embodiments wherein said pulmonary inflammation is associated with acute radiation syndrome.

Preferred methods include embodiments wherein said pulmonary inflammation is caused by a coronavirus.

Preferred methods include embodiments wherein said coronavirus is selected from a group comprising of: a) MERS; b) SARS-CoV-1; and c) SARS-CoV-2.

Preferred methods include embodiments wherein said pulmonary inflammation is a secondary bacterial pneumonia in a subject who is afflicted with a viral infection.

Preferred methods include embodiments wherein in addition to said cord plasma/serum, additional administration of a composition comprising a prophylactically effective amount of a neuraminidase inhibitor is performed.

Preferred methods include embodiments wherein the neuraminidase inhibitor is selected from the group consisting of: oseltamivir phosphate, zanamivir and RJW-270201 (BCX-1812):

Preferred methods include embodiments wherein the neuraminidase inhibitor is oseltamivir phosphate and the composition is administered from a group of administration routes comprising of: a) orally; b) intranasally; c) intravenously, and d) intra-rectally.

Preferred methods include embodiments wherein said method is used to achieve chemoprophlyaxis of pneumonia in a subject who is at risk of developing bacterial pneumonia as a complication of a viral infection comprising administering a prophylactically effective amount of a neuraminidase inhibitor to the subject in addition to cord plasma/serum.

Preferred methods include embodiments wherein the neuraminidase inhibitor is administered within 4 days of the subject's exposure to a host afflicted with a viral infection.

Preferred methods include embodiments wherein the subject is a human selected from the group consisting of: an individual who is at least 50 years old, an individual who resides in a chronic care facility, an individual who has a chronic disorder of the pulmonary or cardiovascular system, an individual who has required regular medical follow-up or hospitalization during the preceding year because of chronic metabolic diseases (including diabetes mellitus), renal dysfunction, hemoglobinopathies, or immunosuppression (including immunosuppression caused by medications or by human immunodeficiency [HIV] virus); an individual between 6 months and 18 years in age who is receiving long-term aspirin therapy, an individual less than 14 years of age, and a woman who will be in the second or third trimester of pregnancy.

Preferred methods include embodiments wherein the subject is a human over 65 years of age.

Preferred methods include embodiments wherein least one antibiotic is selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime, trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine is administered together with cord blood plasma/serum and/or adjuvant.

Preferred methods include embodiments wherein, said adjuvant is administered prior to, and/or concurrently with, and/or subsequently to, said cord plasma and/or serum.

### BRIEF DESCRIPTION OF THE DRAWINGS

FIG. 1 is a bar showing the lung fluid percentage of BALB/c mice that where treated with endotoxin and saline control, cord plasma, and cord plasma+pterostilbene.

### DESCRIPTION OF THE INVENTION

The invention provides the use of umbilical cord blood plasma and/or serum combined with therapeutic adjuvants for treatment of COVID19 induced ARDS as well as prevention of the infection.

This invention is not limited to the particular methodology, protocols, and reagents, etc., described herein and as such can vary. The terminology used herein is for the purpose of describing particular embodiments only and is not intended to limit the scope of the present invention, which is defined solely by the claims. As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.” All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents. Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Disclosed are means of inducing protective state in the lung of an individual susceptible to, or, suffering from acute respiratory distress syndrome (ARDS), especially caused by viral infection, more specifically caused by COVID19. Said protective states are associated with reduced inflammation and pathology of ARDS. The invention teaches that administration of umbilical cord blood together with adjuvants such as pterostilbene which provide an environment conducive to stimulation of cells which inhibit inflammation and stimulate regeneration of damaged pulmonary cells. In one embodiment of the invention, patients are identified as having risk of ARDS based on typical clinical parameters and/or cytokine alterations.

For the practice of the invention, cord blood plasma may be generated through means known in the art. One of the elements of the discovery is that inhibitors of activation of NF-kappa B enhance the protective effect of cord blood plasma in the lung and reduce ARDS pathology.

In one embodiment, cord blood plasma is administered to cells, organs, or organisms in need of rejuvenating activity together with an appropriate concentration of pterostilbene sufficient to activate NRF2 more than 10% compared to baseline.

In some embodiments, cord blood plasma is concentrated by lyophilization or other means known in the art such as filtration, or chromatography, and subsequently administered together with pterostilbene at a concentration of pterostilbene sufficient to activate NRF2.

In another embodiment, cord blood plasma exosomes are administered with pterostilbene. Exosomes are purified and concentrated as described below. Indeed, the applicant has now demonstrated that membrane vesicles, particularly exosomes, could be purified, and possess ability to inhibit pain. In one embodiment, a strong or weak, preferably strong, anion exchange may be performed. In addition, in a specific embodiment, the chromatography is performed under pressure. Thus, more specifically, it may consist of high performance liquid chromatography (HPLC). Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE. POROS®. SEPHAROSE®, SEPHADEX®, TRISACRYL®, TSK-GEL SW OR PW®, SUPERDEX®TOYOPEARL HW and SEPHACRYL®, for example, which are suitable for the application of this invention. Therefore, in a specific embodiment, this invention relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the invention, it is preferable to use supports in bead form. Ideally, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, it is preferable to use high porosity gels, particularly between 10 nm and 5.mu.m, more preferably between approximately 20 nm and approximately 2.mu.m, even more preferably between about 100 nm and about 1.mu.m. For the anion exchange chromatography, the support used must be functionalised using a group capable of interacting with an anionic molecule. Generally, this group is composed of an amine which may be ternary or quaternary, which defines a weak or strong anion exchanger, respectively. Within the scope of this invention, it is particularly advantageous to use a strong anion exchanger. In this way, according to the invention, a chromatography support as described above, functionalised with quaternary amines, is used. Therefore, according to a more specific embodiment of the invention, the anion exchange chromatography is performed on a support functionalised with a quaternary amine. Even more preferably, this support should be selected from poly(styrene-divinylbenzene), acrylamide, agarose, dextran and silica, alone or in mixtures, and functionalised with a quaternary amine. Examples of supports functionalised with a quaternary amine include the gels SOURCEQ. MONO Q, Q SEPHAROSE®, POROS® HQ and POROS® QE, FRACTOGEL®TMAE type gels and TOYOPEARL SUPER®Q gels.

A particularly preferred support to perform the anion exchange chromatography comprises poly(styrene-divinylbenzene). An example of this type of gel which may be used within the scope of this invention isSOURCE 0 gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100.mu.l up to 10 ml or greater. In this way, the supports available have a capacity which may reach 25 mg of proteins/ml, for example. For this reason, a 100.mu.l column has a capacity of approximately 2.5 mg of proteins which, given the samples in question, allows the treatment of culture supernatants of approximately 2 l (which, after concentration by a factor of 10 to 20, for example, represent volumes of 100 to 200 ml per preparation). It is understood that higher volumes may also be treated, by increasing the volume of the column, for example. In addition, to perform this invention, it is also possible to combine the anion exchange chromatography step with a gel permeation chromatography step. In this way, according to a specific embodiment of the invention, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, are preferably used. As an illustration, for gel permeation chromatography, a support such as SUPERDEX®200HR (Pharmacia), TSK G6000 (TosoHaas) or SEPHACRYL® S (Pharmacia) is preferably used. The process according to the invention may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the invention, the biological sample is a culture supernatant of membrane vesicle-producing fibroblast cells.

In addition, according to a preferred embodiment of the invention, the biological sample is treated, prior to the chromatography step, to be enriched with membrane vesicles (enrichment stage). In this way, in a specific embodiment, this invention relates to a method of preparing membrane vesicles from a biological sample, characterized in that it comprises at least: b) an enrichment step, to prepare a sample enriched with membrane vesicles, and c) a step during which the sample is treated by anion exchange chromatography and/or gel permeation chromatography.

In one embodiment, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, a preferred method of preparing membrane vesicles according to this invention more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In an other specific embodiment, the enrichment step comprises an affinity chromatography step, optionally preceded by a step of elimination of cells and/or cell debris (clarification). A preferred enrichment step according to this invention comprises (i) the elimination of cells and/or cell debris (clarification), (ii) a concentration and (iii) an affinity chromatography. The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2.mu.m, e.g. between 0.2 and 10.mu.m, are preferentially used. It is particularly possible to use a succession of filters with a porosity of 10.mu.m, 1.mu.m, 0.5.mu.m followed by 0.22.mu.m.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to a preferred embodiment, the biological sample (e.g., the supernatant) is subjected to an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, G F, Sepracor). Within the scope of the invention, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. Preferably, an affinity chromatography on a dye is used, allowing the elimination (i.e., the retention) of contaminants such as proteins and enzymes, for instance albumin, kinases, deshydrogenases, clotting factors, interferons, lipoproteins, or also co-factors, etc. More preferably, the support used for this chromatography step is a support as used for the ion exchange chromatography, functionalised with a dye. As specific example, the dye may be selected from Blue SEPHAROSE®(Pharmacia), YELLOW 86, GREEN 5 and BROWN 10 (Sigma). The support is more preferably agarose. It should be understood that any other support and/or dye or reactive group allowing the retention (binding) of contaminants from the treated biological sample can be used in the instant invention.

In one embodiment a membrane vesicle preparation process within the scope of this invention comprises the following steps: a) the culture of a population of membrane vesicle (e.g. exosome) producing cell sunder conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In a preferred embodiment, step b) above comprises a filtration of the culture supernatant, followed by an ultrafiltration, preferably tangential. In another preferred embodiment, step b) above comprises a clarification of the culture supernatant, followed by an affinity chromatography on dye, preferably on Blue SEPHAROSE®.

In addition, after step c), the material harvested may, if applicable, be subjected to one or more additional treatment and/or filtration stages d), particularly for sterilization purposes. For this filtration treatment stage, filters with a diameter less than or equal to 0.3.mu.m are preferentially used, or even more preferentially, less than or equal to 0.25.mu.m. Such filters have a diameter of 0.22.mu.m, for example.

After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, a specific preparation process within the scope of the invention comprises at least the following steps: c) an anion exchange chromatography and/or gel permeation chromatography treatment of the biological sample, and d) a filtration step, particularly sterilizing filtration, of the material harvested after stage c). In a first variant, the process according to the invention comprises: c) an anion exchange chromatography treatment of the biological sample, and d) a filtration step, particularly sterilizing filtration, on the material harvested after step c).

In another variant, the process according to the invention comprises: c) a gel permeation chromatography treatment of the biological sample, and d) a filtration step, particularly sterilizing filtration, on the material harvested after step c). According to a third variant, the process according to the invention comprises: c) an anionic exchange treatment of the biological sample followed or preceded by gel permeation chromatography, and d) a filtration step, particularly sterilizing filtration, on the material harvested after step c).

In some embodiments, the composition containing an effective amount of pterostilbene comprises a pharmaceutically acceptable salt of pterostilbene. In some embodiments, the pterostilbene is isolated from a plant material. In some embodiments, the composition contains at least about a 0.75 wt. % pterostilbene component based on the dry weight of the plant isolate. In some embodiments, the pterostilbene is administered daily at a concentration ranging from about 0.007 to about 1500 mg pterostilbene component per kg metabolic weight. In some embodiments, pterostilbene is administered daily at a concentration of about 2.5 mg to about 10 mg of pterostilbene per kilogram of subject body weight. In some embodiments, pterostilbene is administered in capsules at a dose of about 200 mg at least twice daily. In some embodiments, pterostilbene is administered via an extract of Vaccinium berries, such as blueberries. In some embodiments, pterostilbene is administered with a combination of ingredients including, superoxide dismutase, curcumin, DMAE, alpha lipoic acid, and piperine. In some embodiments, the pterostilbene is administered in the form of a sustained release (SR) formulation. In some embodiments, the SR formulation comprises a prodrug, or analog of N-pterostilbene, or a salt or solvate thereof. In some embodiments, the composition, upon oral administration, provides a therapeutically effective plasma concentration of pterostilbene over more than about 3 hours following the administration. In some embodiments, the pterostilbene formulation further comprises an immediate release (IR) component. In some embodiments, the IR component includes a prodrug, or analog of pterostilbene, or a salt or solvate thereof. In some embodiments, the composition, upon oral administration, provides a therapeutically effective plasma concentration of pterostilbene over about 45 minutes to about 20 hours following the administration. In some embodiments, the SR and/or IR component comprises a prodrug or analog of pterostilbene which is less polar than pterostilbene and possesses an increased absorbability profile in the lower gastrointestinal tract of a mammal. In some embodiments, the SR and/or IR component comprises a prodrug of pterostilbene selected from the group consisting of an ester prodrug, an amide prodrug, and an anhydride prodrug.

### Example

BALB/c mice where treated with endotoxin. Specifically, mice were intraperitoneally injected with 50 mg/kg pentobarbital. LPS (5 mg/kg) (Sigma-Aldrich) was delivered to the lungs through a tracheostomy. The weight of the lung was compared to the weight of the body as a measure of lung fluid accumulation.

Mice where administered intravenously control saline, cord plasma (500 uL) or cord plasma plus pterostilbene 5 microMoL at the time of LPS administration. Decreased in lung fluid was observed in the group treated with pterostilbene and cord blood plasma.

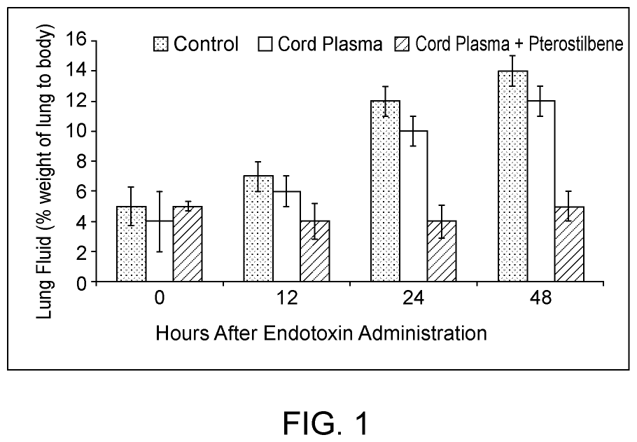

Results are shown in FIG. 1

### REFERENCES

Rincon, F., et al., Impact of acute lung injury and acute respiratory distress syndrome after traumatic brain injury in the United States. Neurosurgery, 2012. 71(4): p. 795-803.
Angus, D. C., et al., Healthcare costs and long-term outcomes after acute respiratory distress syndrome: A phase III trial of inhaled nitric oxide. Crit Care Med, 2006. 34(12): p. 2883-90.
Wilson, J. G. and M. A. Matthay, Mechanical ventilation in acute hypoxemic respiratory failure: a review of new strategies for the practicing hospitalist. J Hosp Med, 2014. 9(7): p. 469-75.
Acute Respiratory Distress Syndrome, N., et al., Ventilation with lower tidal volumes as compared with traditional tidal volumes for acute lung injury and the acute respiratory distress syndrome. N Engl J Med, 2000. 342(18): p. 1301-8.
Zampieri, F. G., et al., Extracorporeal membrane oxygenation for severe respiratory failure in adult patients: a systematic review and meta-analysis of current evidence. J Crit Care, 2013. 28(6): p. 998-1005.
Lee, J. W., Y. Zhu, and M. A. Matthay, Cell-based therapy for acute lung injury: are we there yet? Anesthesiology, 2012. 116(6): p. 1189-91.
Weiss, D. J., Concise review: current status of stem cells and regenerative medicine in lungbiology and diseases. Stem Cells, 2014. 32(1): p. 16-25.
Toonkel, R. L., et al., Mesenchymal stem cells and idiopathic pulmonary fibrosis. Potential for clinical testing. Am J Respir Crit Care Med, 2013. 188(2): p. 133-40.
Lee, J. W., et al., Allogeneic human mesenchymal stem cells for treatment of E. coli endotoxin-induced acute lung injury in the ex vivo perfused human lung. Proc Natl Acad Sci USA, 2009.106(38): p. 16357-62.
Curley, G. F., et al., Effects of intratracheal mesenchymal stromal cell therapy during recovery and resolution after ventilator-induced lung injury. Anesthesiology, 2013. 118(4): p. 924-32.
Mei, S. H., et al., Prevention of LPS-induced acute lung injury in mice by mesenchymal stem cells overexpressing angiopoietin 1. PLoS Med, 2007. 4(9): p. e269.
Xu, J., et al., Mesenchymal stem cell-based angiopoietin-1 gene therapy for acute lung injury induced by lipopolysaccharide in mice. J Pathol, 2008. 214(4): p. 472-81.
Iyer, S. S., et al., Effect of bone marrow-derived mesenchymal stem cells on endotoxin-inducedoxidation of plasma cysteine and glutathione in mice. Stem Cells Int, 2010. 2010: p. 868076.
Danchuk, S., et al., Human multipotent stromal cells attenuate lipopolysaccharide-induced acute lung injury in mice via secretion of tumor necrosis factor-alpha-induced protein 6. Stem Cell Res Ther, 2011. 2(3): p. 27.
Gupta, N., et al., Intrapulmonary delivery of bone marrow-derived mesenchymal stem cells improves survival and attenuates endotoxin-induced acute lung injury in mice. J Immunol, 2007. 179(3): p. 1855-63.
Mei, S. H., et al., Mesenchymal stem cells reduce inflammation while enhancing bacterial clearance and improving survival in sepsis. Am J Respir Crit Care Med, 2010. 182(8): p. 1047-57.
Vallabhaneni, K. C., H. Haller, and I. Dumler, Vascular smooth muscle cells initiate proliferation of mesenchymal stem cells by mitochondrial transfer via tunneling nanotubes. Stem Cells Dev, 2012. 21(17): p. 3104-13.
Kordelas, L., et al., MSC-derived exosomes: a novel tool to treat therapy-refractory graft-versus-host disease. Leukemia, 2014.28(4): p. 970-3.
Lai, R. C., et al., Exosome secreted by MSC reduces myocardial ischemia/reperfusion injury. StemCell Res, 2010. 4(3): p. 214-22.
Zhang, Y., et al., Control of mesenchymal lineage progression by microRNAs targeting skeletalgene regulators Trps1 and Runx2. J Biol Chem, 2012. 287(26): p. 21926-35.

1. A method of treating pulmonary inflammation comprising the steps of: a) identifying a patient with pulmonary inflammation; b) administering cord blood plasma or cord blood plasma and serum together with one or more adjuvants; and c) assessing pulmonary inflammation in order to determine whether adjustment of dosage is necessary.

2. The method of claim 1, wherein said cord blood plasma and serum is derived from umbilical cord at term delivery and is fractionated to obtain fractions enriched in exosomes.

3. The method of claim 1, wherein said one or more adjuvant is an anti-oxidant.

4. The method of claim 3, wherein said anti-oxidant is selected from the group consisting of: ascorbic acid and derivatives thereof, alpha tocopherol and derivatives thereof, rutin, pterostilbene quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, and superoxide dismutase.

5. The method of claim 1, wherein said pulmonary inflammation is caused by a cytokine storm caused by excessive production of cytokines.

6. The method of claim 5, wherein said excessive production of cytokines is mediated by unrestrained activation of monocytes.

7. The method of claim 5, wherein said excessive production of cytokines is production of cytokines selected from the group consisting of: a) MCP-1; b) interleukin 1 beta; c) interleukin 6, d) interleukin 8; e) interleukin 11; f) interleukin-18; g) interleukin-21; h) interleukin 27; i) interleukin 33; j) HMGB-1; and k) TNF-alpha.

8. The method of claim 7, further comprising the administration of hydroxychloroquine and/or chloroquine.

9. The method of claim 7, further comprising the administration of rapamycin.

10. The method of claim 7, further comprising the administration of chelating agent.

11. The method of claim 10, wherein said chelating agent is deferoxamine mesylate.

12. The method of claim 7, further comprising the administration of an agent capable of inhibiting NF-kappa B.

13. The method of claim 12, wherein said NF-kappa B is inhibited by antisense oligonucleotides targeting one or more sequences found in NF-kappa B components.

14. The method of claim 12, wherein said NF-kappa B is inhibited by RNA interference targeting one or more sequences found in NF-kappa B components.

15. The method of claim 14, wherein said RNA interference is induced by administration of short interfering RNA.

16. The method of claim 14, wherein RNA interference is induced by administration of short hairpin RNA.

17. The method of claim 13, wherein said agent capable of inhibiting NF-kappa B activation is a decoy oligonucleotide.

18. The method of claim 12, wherein said agent capable of inhibiting NF-kappa B activation is selected from the group consisting of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, and Saline (low Na+ istonic).

19. The method of claim 1, wherein an immune suppressive agent is further administered to said patient.

20. The method of claim 19, wherein said immune suppressive agent is selected from the group consisting of: cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), Monoclonal antibodies, basiliximab (Simulect), and daclizumab (Zinbryta)
